(19) United States
(12) Patent Application Publication
(10) Pub. No.: US 2011/0213217 A1
(43) Pub. Date: Sep. 1, 2011

(54) ENERGY OPTIMIZED SENSING TECHNIQUES

(75) Inventors: Edward M. McKenna, Boulder, CO (US); Daniel Lisogurski, Boulder, CO (US)

(73) Assignee: Nellcor Puritan Bennett LLC, Boulder, CO (US)

(21) Appl. No.: 12/714,533

(22) Filed: Feb. 28, 2010

(51) Int. Cl. A61B 5/00

(52) U.S. Cl. 600/301

(57) ABSTRACT

The present disclosure describes an energy efficient wireless medical sensor that may be capable of optimizing battery life and increasing component life by selectively using only a subset of the sensors and sensor functionality included in the wireless medical sensor at any one time. One or more update factors may be used by the wireless sensor or an external patient monitor to derive a data collection modality, data collection rates, and update interval. The data collection modality, data collection rates, and update interval may be used to selectively gather sensing data in a manner that is more energy efficient.

### BACKGROUND

The present disclosure relates generally to medical sensors and, more particularly, to wireless medical sensors.

This section is intended to introduce the reader to various aspects of art that may be related to various aspects of the present disclosure, which are described and/or claimed below. This discussion is believed to be helpful in providing the reader with background information to facilitate a better understanding of the various aspects of the present disclosure. Accordingly, it should be understood that these statements are to be read in this light, and not as admissions of prior art.

In the field of medicine, doctors often desire to monitor and sense certain physiological characteristics of their patients. Accordingly, a wide variety of devices have been developed for monitoring and sensing many such physiological characteristics. For example, one category of monitoring and sensing devices includes spectrophotometric monitors and sensors. This category of device studies the electromagnetic spectra (e.g., wavelengths of light) and can monitor a suite of physiological parameters. Such devices provide doctors and other healthcare personnel with the information they need to provide the best possible healthcare for their patients. As a result, such monitoring and sensing devices have become an indispensable part of modern medicine.

Conventional spectrophotometric sensors are typically connected to a monitor via a cable. The cable provides the sensor with power and acts as a conduit for the transmission of signals between the sensor and the monitor. However, the cable also acts to tether the patient to the monitor, preventing unencumbered motion by the patient. As a result, such cable-based systems may not be suitable for ambulatory patients or for applications that require remote monitoring in non-clinical environments. Accordingly, various systems have been proposed which include a patient sensing device connected to a local monitor by way of a wireless link. Unfortunately, sensors that incorporate a wireless link may be limited to that power which is provided on the sensor itself, which may be drained very quickly.

### BRIEF DESCRIPTION OF THE DRAWINGS

Advantages of the disclosed techniques may become apparent upon reading the following detailed description and upon reference to the drawings in which:

FIG. 1 illustrates components of a wireless medical system, in accordance with one embodiment of the present disclosure;

FIG. 2 depicts a diagram of a wireless sensor and monitor, in accordance with one embodiment of the present disclosure;

FIG. 3 is a flowchart describing an embodiment of a method for sensing data using the system of FIG. 1, in accordance with an embodiment;

FIG. 4 is a schematic diagram of various update factors that may be employed with the method of FIG. 3, in accordance with an embodiment;

FIG. 5 is a communication diagram illustrating communication between a wireless sensor and a patient monitor of the system of FIG. 1, in accordance with an embodiment, and;

FIG. 6 is a flowchart describing an embodiment of another method for sensing data using the system of FIG. 1, in accordance with an embodiment.

### DETAILED DESCRIPTION OF SPECIFIC EMBODIMENTS

One or more specific embodiments of the present techniques will be described below. In an effort to provide a concise description of these embodiments, not all features of an actual implementation are described in the specification. It should be appreciated that in the development of any such actual implementation, as in any engineering or design project, numerous implementation-specific decisions must be made to achieve the developers' specific goals, such as compliance with system-related and business-related constraints, which may vary from one implementation to another. Moreover, it should be appreciated that such a development effort might be complex and time consuming, but would nevertheless be a routine undertaking of design, fabrication, and manufacture for those of ordinary skill having the benefit of this disclosure.

Present embodiments relate to systems, methods, and devices for balancing power consumption and lifespan of medical sensors systems. For example, a medical sensor system may include a sensor device, a patient monitor, and wireless transmission circuitry. The sensor device may be capable of obtaining a sensor measurement from a patient and of sampling the measurement at certain data collection rates to obtain discrete values. Further, the sensor device and/or the patient monitor may also be capable of determining certain sensor settings based at least in part on update factors associated with a status of the patient, and the wireless transmission circuitry may be capable of wirelessly transmitting and receiving data between the sensor device and the monitoring device. Because the embodiments presently disclosed may reduce the amount of time that the sensor device spends in sensing the patient, the sensor device may expend less power and, accordingly, may have a longer battery life and overall lifespan.

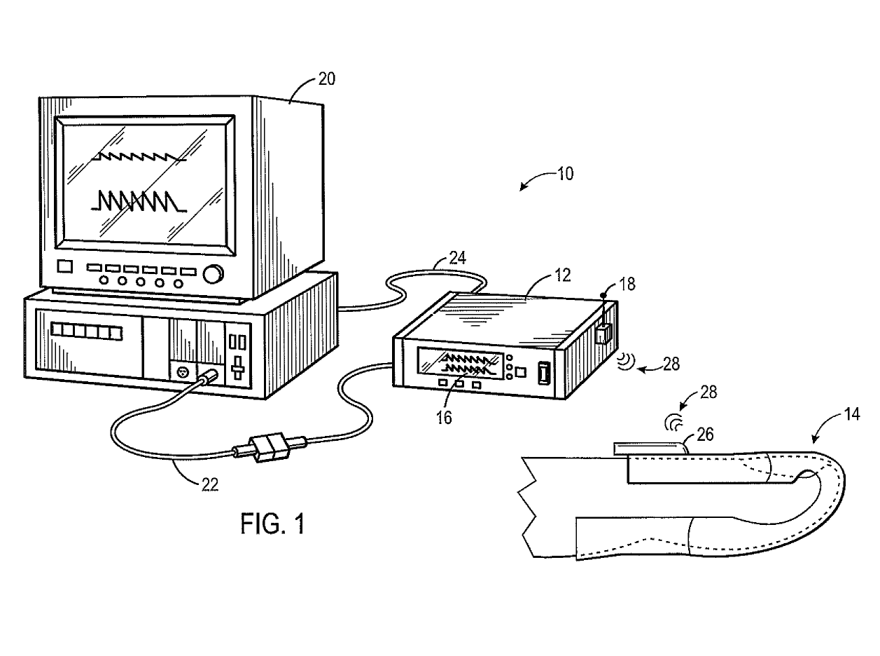

With the foregoing in mind, FIG. 1 depicts an embodiment of a medical system 10 that may efficiently sense patient status, thereby conserving power. Although the embodiment of the system 10 illustrated in FIG. 1 relates to photoplethysmography, the system 10 may be configured to obtain a variety of medical measurements with a suitable medical sensor. For example, the system 10 may, additionally or alternatively, be configured to measure patient temperature, transvascular fluid exchange volumes, tissue hydration, blood flow, blood microcirculation, bilirubin levels, non-invasive blood pressures (NIBP), and/or a pulse transit time, and so forth. The system 10 may include a patient monitor 12 that communicates wirelessly with a wireless sensor 14. One or more additional subsensors may also be present in the wireless sensor 14. It is to be understood that the term “subsensor” is used herein to denote any type of sensor or sensing modality that may be included as a component of the wireless sensor 14. Some subsensors include, for example, a Doppler subsensor that may be used to measure a blood flow at a patient tissue site, a photoacoustic subsensor that may be used for measuring microcirculation, a photon density subsensor that may be used for measuring various blood-related parameters, and so forth. Indeed, a wide variety of sensors may be included as subsensor components of the wireless sensor 14.

The patient monitor 12 may include a display 16, a wireless module 18 for transmitting and receiving wireless data, a memory, a processor, and various monitoring and control features. Based on subsensor data received from the wireless sensor 14, the patient monitor 12 may display patient measurements and perform various measurement or processing algorithms. For example, when the system 10 is configured for pulse oximetry, the patient monitor 12 may perform blood oxygen saturation calculations, pulse measurements, and other measurements based on the received wireless sensor 14 data. Furthermore, to upgrade conventional operation provided by the monitor 12 to provide additional functions, monitor 12 may be coupled to a multi-parameter patient monitor 20 via a cable 22 connected to a sensor input port or via a cable 24 connected to a digital communication port, for example.

In one embodiment of the system 10, the wireless sensor 14 may include a pulse oximeter subsensor. However, the sensor 14 may also include other types of patient subsensors, capable of obtaining any of a variety of medical measurements. For example, as discussed above, the wireless sensor 14 may include subsensors capable of measuring patient temperature, transvascular fluid exchange volumes, tissue hydration, blood flow, pulse rate, respiration, bilirubin levels, non-invasive blood pressures (NIBP), and/or a pulse transit time, and so forth. As described further below, in one embodiment the wireless sensor 14 may collect subsensor measurements, such as a light intensity for pulse oximetry applications, patient temperature, and others, based in part on certain sensor settings that depend on one more or more update factors relating to the status of a patient. By selectively collecting subsensor measurements from one or more subsensors, the sensor 14 may optimize the battery usage and improve the lifespan of sensor components.

Like the patient monitor 12, the wireless sensor 14 may include a wireless module 26. The wireless module 26 of the wireless sensor 14 may establish a wireless communication 28 with the wireless module 18 of the patient monitor 12 using any suitable protocol. By way of example, the wireless module 26 may be capable of communicating using the IEEE 802.15.4 standard, and may communicate, for example, using ZigBee, WirelessHART, or MiWi protocols. Additionally or alternatively, the wireless module 26 may be capable of communicating using the Bluetooth standard or one or more of the IEEE 802.11 standards.

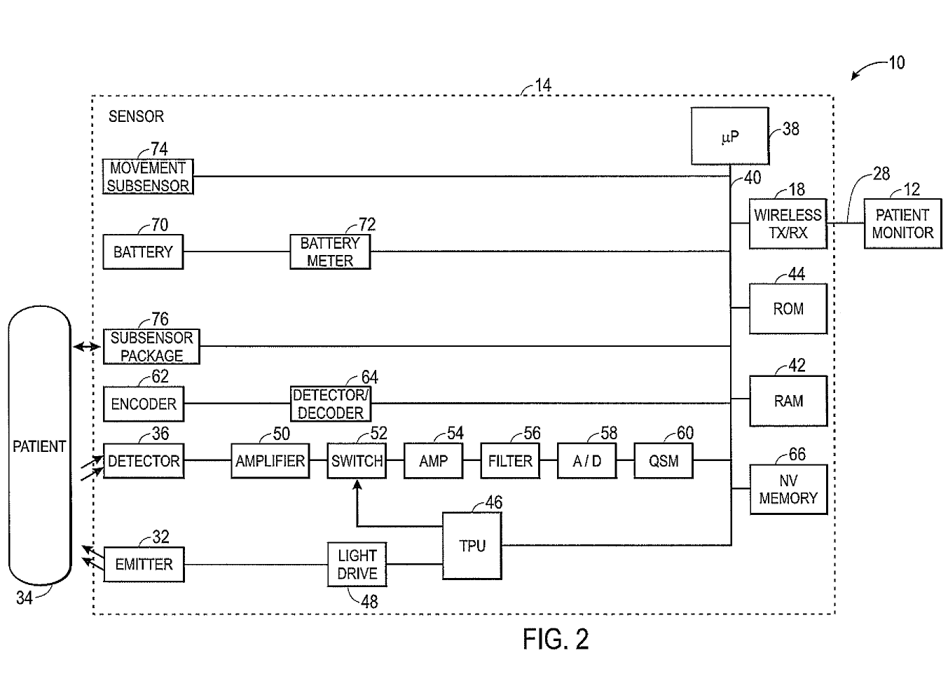

FIG. 2 is a block diagram of an embodiment of the wireless medical sensor system 10 that may be configured to implement the techniques described herein. By way of example, embodiments of the system 10 may be implemented with any suitable medical sensor and patient monitor, such as those available from Nellcor Puritan Bennett LLC. The system 10 may include the patient monitor 12 and the wireless sensor 14, which may be configured to obtain, for example, a plethysmographic signal from patient tissue at certain predetermined wavelengths. The wireless sensor 14 may be communicatively connected to the patient monitor 12 via wireless communication 28 (shown in FIG. 1). When the system 10 is operating, light from an emitter 32 may pass into a patient 34 where the portions of the light may be differentially scattered, absorbed, and/or transmitted. Light that emerges from the patient tissue may detected by a detector 36.

The sensor 14 may include a microprocessor 38 connected to an internal bus 40. Also connected to the bus 40 may be a RAM memory 42 and a ROM memory 44. A time processing unit (TPU) 46 may provide timing control signals to a light drive circuitry 48 which may control when the emitter 32 is illuminated, and if multiple wavelengths are emitted, the multiplexed timing for the different wavelengths. The TPU 46 may also control the gating-in of signals from the detector 36 through an amplifier 50 and a switching circuit 52. These signals may be sampled at the proper time, depending upon which of multiple wavelengths of light is emitted, if multiple wavelengths are used. In one embodiment, the received signal from the detector 36 may be passed through an amplifier 54, a low pass filter 56, and/or an analog-to-digital converter 58.

The digital data may then be stored in a queued serial module (QSM) 60, for later downloading to the RAM 42 as the QSM 60 fills up. In one embodiment, there may be multiple parallel paths of separate amplifier, filter and/or A/D converters for multiple light wavelengths or spectra received. This raw digital data may be further processed by the circuitry of the wireless medical sensor 14 into specific data of interest, such as pulse rate, blood oxygen saturation, and so forth.

In an embodiment, the sensor 14 may also contain an encoder 62 that encodes information indicating the wavelength of one or more light sources of the emitter 32, which may allow for selection of appropriate calibration coefficients for calculating a physiological parameter such as blood oxygen saturation. The encoder 62 may, for instance, be a coded resistor, EEPROM or other coding devices (such as a capacitor, inductor, PROM, RFID, parallel resonant circuits, or a colorimetric indicator) that may provide a signal to the processor 38 related to the characteristics of the wireless sensor 14 that may allow the processor 38 to determine the appropriate calibration characteristics for the photoplethysmographic components of the wireless sensor 14. Further, the encoder 62 may include encryption coding that prevents a disposable or replaceable part of the wireless sensor 14 from being recognized without corresponding adjustment or replacement of the information encoded by the encoder 62. In some embodiments, the encoder 62 and/or the detector/decoder 64 may not be present. Additionally or alternatively, the processor 38 may encode processed sensor data before transmission of the data to the patient monitor 12.

In various embodiments, based at least in part upon the value of the received signals corresponding to the light received by detector 36, the microprocessor 38 may calculate a physiological parameter of interest using various algorithms. These algorithms may utilize coefficients, which may be empirically determined, corresponding to, for example, the wavelengths of light used. These may be stored in the ROM 44 or non-volatile memory 66. In a two-wavelength system, the particular set of coefficients chosen for any pair of wavelength spectra may be determined by the value indicated by an encoder 62 corresponding to a particular light source of the emitter 32. For example, the first wavelength may be a wavelength that is highly sensitive to small quantities of deoxyhemoglobin in blood, and the second wavelength may be a complimentary wavelength. Specifically, for example, such wavelengths may be produced by orange, red, infrared, green, and/or yellow LEDs. Different wavelengths may be selected based on instructions or protocols received from the patient monitor 12, based on preferences stored in a nonvolatile storage 66, or based on user input. The instructions from the patient monitor 12 may be transmitted wirelessly to the sensor 14 and may be selected at the patient monitor 12 by a user interface provided as part of the patient monitor 12 or via a port providing instructions from a remote host computer.

A nonvolatile memory 66 may store caregiver preferences, patient information, or various parameters, discussed below, which may be used in the operation of the sensor 14. Software for performing the configuration of the sensor 14 and for carrying out the techniques described herein may also be stored on the nonvolatile memory 66 and/or on the ROM 44. The nonvolatile memory 66 and/or RAM 42 may also store historical values of various discrete medical data points. By way of example, the nonvolatile memory 66 and/or RAM 42 may store values of instantaneous pulse rate for every second of the most recent five minutes.

A battery 70 may supply the wireless medical sensor 14 with operating power. By way of example, the battery 70 may be a rechargeable battery, such as a lithium ion or lithium polymer battery, or may be a single-use battery such as an alkaline or lithium battery. Due to the techniques described herein to reduce battery consumption, the battery 70 may be of a much lower capacity, and accordingly much smaller and/or cheaper, than a battery needed to power a similar wireless sensor 14 that does not employ these techniques. A battery meter 72 may provide the expected remaining power of the battery 70 to a user and/or to the microprocessor 38. The remaining battery life indicated by the battery meter 72 may be used as a factor in determining the update interval, as discussed in greater detail below.

The wireless sensor 14 may also include a movement subsensor 74, such as one or more accelerometers, that may sense when the patient 34 moves the wireless sensor 14. Whether the patient is at rest or moving, as indicated by the movement subsensor 74, may also be used as a factor in determining the wireless sensor's 14 data collection modality (i.e., how many subsensors in the wireless sensor 14 are activated and the subsensor functions used), the data collection rate for each subsensor in the wireless sensor 14, and the update interval for reconfiguring the wireless sensor 14 settings, as discussed in greater detail below. In certain embodiments, a package of subsensors 76 may be included for performing specific medical measurements, for example, for measuring or monitoring temperature, photoacoustics, pressure, pH, Doppler shift, photon density, and others.

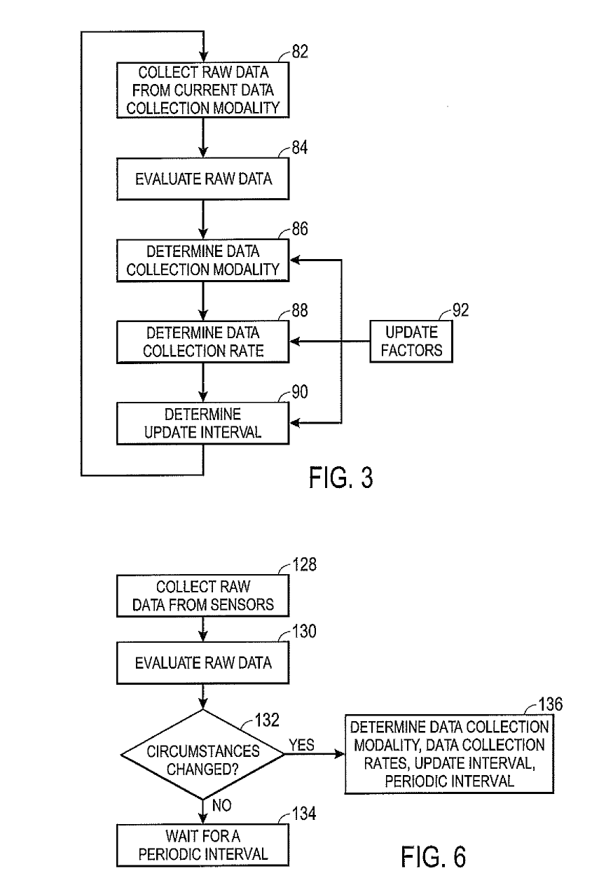

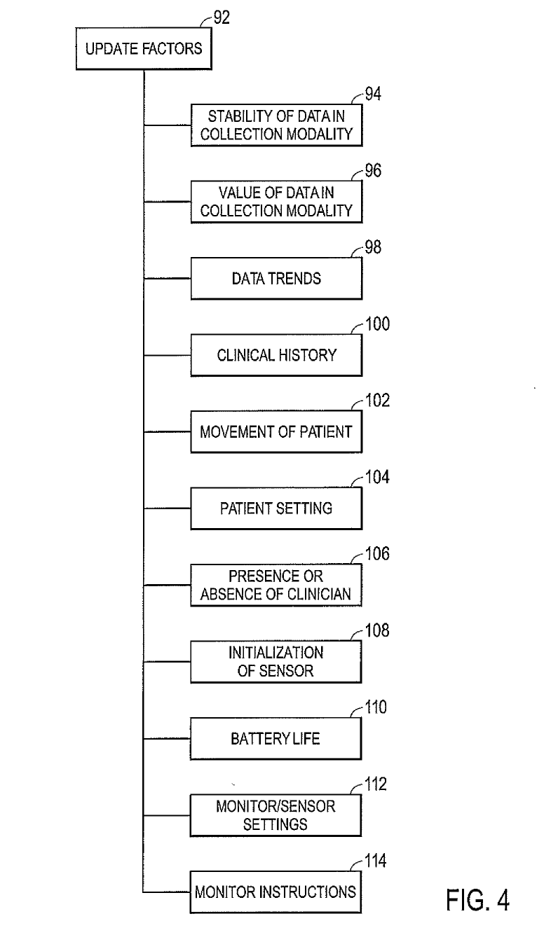

To conserve the amount of power used by the wireless sensor 14, the microprocessor 38 may set the data collection modality, the data collection rate for each one of the subsensors in the data collection modality, and the update interval for reconfiguring the wireless sensor's 14 settings, as described in greater detail below. The microprocessor 38 may carry out these techniques based on instructions executed by the microprocessor 38 and stored in the RAM 42, the ROM 44, the nonvolatile memory 66, and/or based on instructions received from the patient monitor 12. Specifically, because continuously sensing data from all the subsensors may consume a substantial amount of power, several or all subsensors may generally remain deactivated during a specific update interval. Depending on the current data collection modality, only some of the different subsensors may be temporarily activated. Because the subsensors may only be in use at specific times, less power may be consumed and the life of the battery 70 may be extended. In selecting which of the subsensors to activate/deactivate, the subsensor functions to use, the data collection rate to use for each subsensor, and the update interval, the microprocessor 38 and/or the monitor 12 may consider a variety of update factors including the subsensor data currently being obtained from the patient 36 by the active subsensors in the current data collection modality 14. These various update factors are described in greater detail below with reference to FIGS. 3, 4.

FIG. 3 illustrates a flowchart describing an embodiment of a method that may be implemented by the microprocessor 38 and/or the monitor 12 to efficiently use energy by turning on and utilizing only a subset of the subsensors in the wireless sensor 14 during an update interval. Each one of the subsensors in the wireless sensor 14 may have one or more different sensing functions. For example, a spectrophotometric subsensor may have functions to sense pulse rate, SpO₂, and others. The method described by the flowchart may derive which one of the subsensors in the wireless sensor 14 to use (i.e., flag as active) as well as which one of the sensing functions in each active subsensor to use. The subset of subsensors that have been flagged as active and the sensing functions that will be used is known as a “data collection modality”. The method described by the flowchart may also establish the data collection rate for each subsensor in the wireless sensor 14 and a new update interval which specifies the time interval to wait before updating the data collection modality and the data collection rate for each subsensor. Thus, the amount of time spent in sensing activities may be significantly reduced as compared to continuously sensing and processing data using all available sensing components. Accordingly, the consumption of battery power as well as the wear on sensor components may be significantly reduced.

As depicted in the flowchart of FIG. 3, the wireless sensor 14 may collect (block 82) raw data from all the subsensors 32, 36, 74, 76 participating in the current data collection modality. For example, a current collection modality may include subsensors and sensing functionality that measure temperature, pulse rate, and arterial oxygen saturation. All other subsensors that are not members of the current data collection modality are turned off and are deemed non-participating subsensors. Raw data being received from the subsensors in the current collection modality is evaluated (block 84). The evaluation of the raw data may be performed by the wireless sensor's 14 microprocessor 38 or the raw data may be wirelessly communicated to the monitor 12 for evaluation by the monitor 12. The microprocessor 38 and/or the monitor 12 may first convert the raw data into discrete, meaningful points of data based on the sensing functions selected as part of the current data collection modality. For example, the microprocessor 38 and/or the monitor 12 may process a raw photoplethysmographic data set and derive a pulse rate, respiration rate data, blood oxygen saturation data, etc.

Such discrete data may then be evaluated with respect to certain update factors 92 of FIG. 4 and used to determine (block 86) or update the data collection modality. In some embodiments it may be advantageous to have the monitor 12 evaluate some or all of the discrete data because the monitor 12 may have more processing power, resulting in faster and more powerful computational abilities for the medical system. In other embodiments it may be advantageous to have the microprocessor 38 evaluate some or all of the discrete data because these embodiments may allow for increased patient mobility to locations where the wireless sensor 14 and the monitor 12 may not be communicatively connected. In yet other embodiments it may be advantageous to have both the microprocessor 38 and the monitor 12 evaluate some or all of the discrete data because these embodiments allow for a sharing of the computational burden between the wireless sensor 14 and the monitor 12.

The microprocessor 38 and/or the monitor 12 may maintain the current data collection modality or may determine a new data collection modality (i.e., a new group of subsensors that will be flagged as active as well as the sensing functions from each subsensor that will be used) based on the discrete data derived at step 84, the current data collection modality, the update factors 92 of FIG. 4, and the particular medical application for which the sensor 14 is used. Any number of suitable update factors 92 may be considered, many of which may be described with reference to FIG. 4 below. By way of example, if during patient monitoring the current data collection modality is a temperature modality (i.e., temperature sensing only) and the temperature has remained stable over a recent historical period (e.g., 5 minutes) the next data collection modality may remain a temperature modality. However, if the measured temperature has changed beyond a predetermined threshold then the next collection modality may include subsensors and sensing functions for measuring temperature and pulse rate.

Thus, in one embodiment, the updated data collection modality may escalate the number of subsensors placed in service (i.e., flagged as active subsensors) and/or the increase the amount of data processing (i.e., add more sensing functions) if the currently monitored conditions cross a threshold. For example, the first collection modality (i.e. when the wireless sensor 14 is first turned on) may typically have only a single active subsensor, for example, a temperature subsensor sensing patient temperature. As the patient temperature changes beyond a predetermined threshold, the next collection modality may activate a second subsensor, for example, a pulse oximetry subsensor which may derive pulse rate using a single LED in addition to using the temperature subsensor to sense temperature. A further change in the heart rate of the patient triggering another set of thresholds may result in the next collection modality turning on a second LED in the pulse oximetry subsensor for calculating SpO₂, in addition to the pulse rate and temperature sensing. Conversely, if measured parameters return to a normal range, various subsensors on the wireless sensor 14 that had been activated might be deactivated. While the above scenario describes one implementation in which the wireless sensor 14 initially activates a single subsensor and progressively activates additional subsensors if the measured parameters are determined to be outside of prescribed tolerance ranges, in a different implementation the wireless sensor 14 may initially activate all or almost all available subsensors and progressively deactivate subsensors if the measured parameters are determined to be in prescribed tolerance ranges. Accordingly, the medical system is able to more efficiently use available power and increase the lifecycle of the system's components by not continuously maintaining the subsensors and sensing functions in an active state.

The microprocessor 38 and/or the monitor 12 may determine (block 88) a new data collection rate for each subsensor type based on the specified collection modality, and the update factors 92, some which are shown in FIG. 4. The data collection rate determines the rate at which the respective active subsensors of the data collection modality collect data. A faster data collection rate will result in more data points being collected but will also increase the energy usage of the wireless sensor 14. Accordingly, the data collection rate that may result in an efficient balance between the need to observe the patient and the need to conserve battery power may be determined. Typically, a faster data collection rate may be used as the patient condition changes beyond certain predetermined thresholds or if there are indications that the patient condition is changing. By way of example, if the SpO₂ calculations derived from the spectrophotometric subsensors have remained stable over the recent historical period (e.g., 5 minutes) then the data collection rate may remain unchanged. However, if the SpO₂ calculations show, for example, a decreasing SpO₂ value, then the data collection rate may be increased so that readings of arterial oxygen saturation are taken more often. It is to be understood that the threshold values used to increase or decrease the data collection rate may depend on the type of subsensor used and the particular type of medical application for which the wireless sensor 14 is being used. For example, cardiac monitoring applications may require that a pulse oximetry subsensor collect data every few milliseconds while temperature monitoring applications may require that the temperature subsensors collect data every few seconds or minutes.

The microprocessor 38 and/or the monitor 12 may determine (block 90) a new update interval based on the specified data collection modality and data collection rate. The determined update interval may specify the length of time the data collection modality and data collection rate are employed before the suitability of the modality and rate are again evaluated. For example, an update interval of 3 minutes would result in the wireless sensor 14 collecting data and processing data from all the subsensors in the current data collection modality for three minutes at the respective specified rates. At the end of the update interval, e.g. three minutes, the wireless sensor 14 may reevaluate the data collected from the current collection modality and select or leave unchanged the data collection modality, data collection rate, and/or update interval. In one embodiment where the update interval is derived by the monitor 12, the monitor 12 may be constantly evaluating the data collection modality and/or the data collection rate so as to optimize data gathering activities and wireless sensor 14 energy use. In this embodiment, the update interval duration may be very small, consisting of a few CPU cycles.

In certain other embodiments where the update interval is derived by the monitor 12 and/or the microprocessor 38, the monitor 12 and/or the microprocessor 38 may establish longer update interval. In these embodiments the update interval may have a longer duration in order to, for example, save on energy usage. The update interval in these embodiments may be predetermined or may be derived based on the current data collection modality, the data collection rate, and/or the update factors 92 discussed herein. The monitor 12 and/or the microprocessor 38 may lengthen the update interval in situations where the current data readings are stable and shorten the update interval in situations where the current data readings are changing. By way of example, if the SpO₂ calculations derived from the spectrophotometric subsensor have remained stable over the recent historical period (e.g., 5 minutes) then the update interval may remain unchanged. However, if the SpO₂ calculations show, for example, a decreasing SpO₂ value, then the update interval may be decreased so that the evaluations of the optimum data collection modality and the optimum data collection rate may be made more often. The process depicted in FIG. 3 may be performed iteratively during the length of time the wireless sensor 14 is worn by the patient.

As mentioned above, the microprocessor 38 and/or the monitor 12 may use a set of update factors 92 in determining the data collection modality, the data collection rate, and/or the update interval. FIG. 4 illustrates a block diagram of many such update factors 92. As should be appreciated, precisely which update factors 92 are considered by the microprocessor 38 may be predetermined, may be determined based on the current subsensor package (i.e. based on all the subsensors included in a wireless sensor 14 device), may be determined based on the current patient condition, and/or may be determined based on the particular medical application for which the wireless sensor 14 is being used.

One example of an update factor may be the stability 94 of the data received by the active subsensors in the current data collection modality. As mentioned above, a data collection modality may flag as active any number of subsensors that are included in the wireless sensor device 14 shown in FIG. 2. The current data collection modality may be configured to use various sensing functions to sense patient conditions such as temperature, respiration rate, pulse rate, pulse transit time, blood pressure, pH, blood microcirculation, blood flow, and/or SpO₂, among others. If the data from the subsensors in the current data collection modality is within a predetermined variability or standard deviation threshold over a recent historical period (e.g., 5 minutes), the stability factor 94 may weigh in favor of a lower data collection rate, a longer update interval, and/or towards the exclusion of some of the subsensors and sensing functionality used in sensing patient condition from the next data collection modality.

For example, if the patient's heart rate is varying, going up and then coming down repeatedly, then the heart rate data would be deemed unstable. Accordingly, the factor 94 may weigh in favor of a higher data collection rate, a shorter update interval, and/or towards the addition of more subsensors and sensing functionality for sensing the patient condition. In determining the data collection modality, data collection rate, and/or update interval based at least in part on the factor 94, a further consideration may be how much the subsensor data has varied. For example, the greater the variability of the subsensor data, the more the factor 94 may weigh in favor of a higher data collection rate, a shorter update interval, and/or towards the inclusion of more subsensors and sensing functionality in the next data collection modality.

A second example of an update factor 92 may be the actual values 96 derived from the raw data from each subsensor in the current data collection modality (e.g., patient temperature, pulse rate, respiration rate, and others). If a value is within an acceptable range of values, this factor may weigh in favor of a lower data collection rate, a longer update interval, and/or towards the exclusion of some of the subsensors and sensing functions sensing the patient condition from the next data collection modality.

By way of example, if the measured values include a temperature value, an acceptable range of values for an adult patient may be a range of 96.4° F. to 100.4° F. A temperature value less than 96.4° F. or greater than 100.4° F. may be evaluated by the microprocessor or the monitor as weighing in favor of a higher data collection rate, a shorter update interval, and/or towards the addition of more subsensors and sensing functionality sensing the patient condition. In determining the data collection modality, the data collection rate, and/or the update interval based at least in part on the values 96, the microprocessor 38 or the monitor 12 may further consider how much the value of the data of interest is outside or inside the acceptable range. For example, the greater the extent by which the value is outside the acceptable range, the more the value factor 96 may weigh in favor of a higher data collection rate, a shorter update interval, and/or towards the addition of more subsensors and sensing functions sensing the patient condition.

A third example of an update factor 92 may be data trends 98 observed in data measured by the various subsensors. For example, if the patient pulse rate is going up and the patient systolic/diastolic pressure is also going up, then this data trend combination may weigh in favor of a higher data collection rate, a shorter update interval, and/or towards the addition of other subsensors and sensing functionality for sensing the patient condition. Similarly, if measured data derived using subsensors in the current data collection modality show a trend towards normalcy of the data, then the data trend may weigh in favor of a lower data collection rate, a longer update interval, and/or towards the exclusion of some of the subsensors and sensing functionality from the next data collection modality.

Data trends 98 may also include trends based on the rate of change for a given parameter. For instance, heart rate may change relatively quickly while body temperature may take longer to show a measurable change. Accordingly, each subsensor may monitor the rate of change of a plurality of physiological parameters. If the rate of change of a parameter trends toward normalcy, then the data trend may weigh in favor of a lower data collection rate, a longer update interval, and/or towards the exclusion of some of the subsensors and sensing functionality from the next data collection modality. A rate of change outside of normalcy for the parameter may weigh in favor of a higher data collection rate, a shorter update interval, and/or towards the addition of other subsensors and sensing functionality for sensing the patient condition. Additionally, each subsensor may further optimize energy usage by including minimum and maximum data collection and/or data update rates based on, for example, the expected rate of change of the physiological parameters under observation. That is, the data collection and data update rates may be adjusted to correspond to expected rates of change of the physiological parameter being monitored. For example, body temperature may change relatively slowly; accordingly, it may be more efficient to sample temperature data at relatively low data collection and data update rates. Heart rate may change relatively quickly; accordingly, better monitoring may be obtained by sampling data at relatively high data collection and data update rates.

A fourth example of an update factor 92 may be the patient's clinical history 100. The clinical history 100 (e.g., patient ages, sex, the treatment or procedure prescribed, the diagnosis, existing medical conditions or previous treatments received, and so forth) may be electronically entered in an external computer (such as an electronic hospital or medical record), communicated to the monitor 12 and or wireless sensor 14, and used as an update factor. The clinical history 100 (such as the patient's age, recent medical procedures, and so forth) may weigh in favor of a higher or lower data collection rate, a longer or shorter update interval, and/or towards the addition or removal of subsensors and sensing functionality for sensing the patient condition.

A fifth example of an update factor 92 may be the movement 102 of the patient, which may be indicated to the wireless medical sensor 14 via parameter updates from the patient monitor 12 or via one or more movement subsensors 74 disposed within the wireless sensor 14. If the patient 34 is currently moving or has undergone recent movement, indicating that the patient 34 is not at rest or is being moved from one room to another, the movement 102 may weigh in favor of a higher data collection rate, a shorter update interval, and/or towards the addition of other subsensors and sensing functionality for sensing the patient condition. If the patient is not currently moving or has not undergone recent movement, the movement 102 may weigh in favor of a lower data collection rate, a longer update interval, and/or towards the exclusion of some of the subsensors and sensing functionality from the next data collection modality. In one embodiment, the amount (or absence) of patient movement 102 may affect the extent to which the data collection rate, the update interval, and/or the addition or removal of other subsensors and sensing functionality for sensing the patient condition is adjusted.

A sixth example of an update factor 92 may be the current location 104 of the patient, which may be determined by a user input, by a RFID tag (or other location determining component) provided with the wireless sensor 14, and/or by a network or wireless communication node accessed by the wireless sensor 14. For example, it may be desirable that the wireless sensor 14 generate different types or amounts of patient data depending on whether the patient 34 is in surgery, in recovery, or undergoing other diagnostic tests. Thus, the current location of the patient may be considered as one of the update factors 92. For example, if the patient is currently located in a medical facility room where the patient should be kept under especially close scrutiny, such as an operating room, the setting 104 may weigh in favor of a higher data collection rate, a shorter update interval, and/or towards the addition of other subsensors and sensing functionality for sensing the patient condition. If the patient is currently located in a medical facility room where the patient 34 may be kept under less scrutiny, such as a waiting or pre-operative room, the setting 104 may weigh in favor of a lower data collection rate, a longer update interval, and/or towards the exclusion of some of the subsensors and sensing functionality from the next data collection modality.

In determining the data collection modality, the data collection rate, and/or the update interval based at least in part on the setting 104, the microprocessor 38 and/or monitor 12 may give different locations different weights in favor of the inclusion or exclusion or certain subsensors and sensing functionality, a shorter or longer data collection rate, and a shorter or longer update interval. For example, if the current location is an operating room or a pre- or post-operative setting, the setting 104 may weigh in favor of a higher data collection rate, a shorter update interval, and/or towards the addition of other subsensors and sensing functionality for sensing the patient condition. However, the setting 104 may weigh more heavily in favor of a lower data collection rate, a longer update interval, and/or towards the exclusion of some of the subsensors and sensing functionality from the next data collection modality if the current location of the patient is a waiting room or a room where diagnostic testing or imaging is performed.

A seventh example of an update factor 92 may be the presence or the absence (block 106) of a clinician or other caregiver proximate to the patient. Such information may be provided as an input to the monitor 12 or wireless sensor 14 when the clinician or caregiver logs into a monitor 12 or when the clinician or caregiver is identified as being in the vicinity of the wireless sensor 14, such as by accessing or entering a room or wing using an access card. For example, if a clinician enters a room where the patient is currently located, the presence 106 of the clinician may weigh in favor of a higher data collection rate, a shorter update interval, and/or towards the addition of other subsensors and sensing functionality for sensing the patient condition. If the clinician exits the room, the absence 106 of the clinician may weigh in favor of a lower data collection rate, a longer update interval, and/or towards the exclusion of some of the subsensors and sensing functionality.

In determining the update interval based at least in part on the presence or absence 106 of a clinician or caregiver, the microprocessor 38 and/or the monitor 12 may weigh the presence or absence 106 based on the number or patient assignment of clinicians present. For example, if a clinician that is not assigned to the patient enters a room where the patient is currently located, the presence of the unassigned clinician may not weigh as heavily in favor of a higher data collection rate, a shorter update interval, and/or towards the addition of other subsensors and sensing functionality for sensing the patient condition as when a clinician that is assigned to the patient enters the room. Thus, the presence or absence 106 factor may take into account who the individual is who is present or absent, their relationship or assignment to the patient, their training or designation (i.e., doctor, nurse, therapist, and so forth), as well as other factors that may be useful in assessing the import of having such a person present or absent with respect to care of the patient.

An eight example of an update factor 92 may be an initialization status 108 of the wireless sensor 14. For a period of time while the sensor 14 is being initialized (e.g., 5 minutes), the number of subsensors and sensing functionality in the current data collection modality, the data collection rate and/or the update interval of the wireless sensor 14 may be temporarily increased, and the raw data stream supplied to the patient monitor 12. By supplying a raw data stream during the initialization of the wireless sensor 14, a clinician for other medical personnel may properly fit the wireless sensor 14 to the patient. In this way, the sensor initialization 108 may weigh in favor of a high data collection rate, a short update interval, and/or towards the addition of all the subsensors and sensing functionality for sensing the patient condition when the wireless sensor 14 has recently been activated.

A ninth example of an update factor 92 may be the battery life 110 of the wireless sensor 14. If the battery 70 of the wireless sensor 14 has more than a predetermined amount of remaining battery life, the battery life 110 may weigh in favor of a higher data collection rate, a shorter update interval, and/or towards the addition of other subsensors and sensing functionality for sensing the patient condition. If the battery 70 has less than the predetermined amount of remaining battery life 110, this may weigh in favor of a lower data collection rate, a longer update interval, and/or towards the exclusion of some of the subsensors and sensing functionality. In some embodiments, there may not be a single threshold, but may instead be two or more thresholds or a dynamic function or relationship that relates battery life 110 to the data collection rate, the update interval, and/or the number or type of subsensors activated. Further, to the extent that not all of the subsensor types included on the wireless sensor 14 may consume power at the same rate, the power consumption of the various subsensor types may be considered in conjunction with battery life 110 in adjusting the data collection rate, the update interval, and/or the specific subsensors activated.

A tenth example of an update factor 92 may be the monitor and/or sensor settings 112. One or both of the monitor 12 and/or the wireless sensor device 14 may include user-selectable settings such as the data collection rate for individual subsensors, the set of subsensors and sensing functionality to include in the data collection modality, and/or the length of the update interval. These settings may be stored in the monitor 12 and/or the wireless sensor device 14 and may be configured by a medical professional for specific patients, medical applications, and/or patient settings, among others.

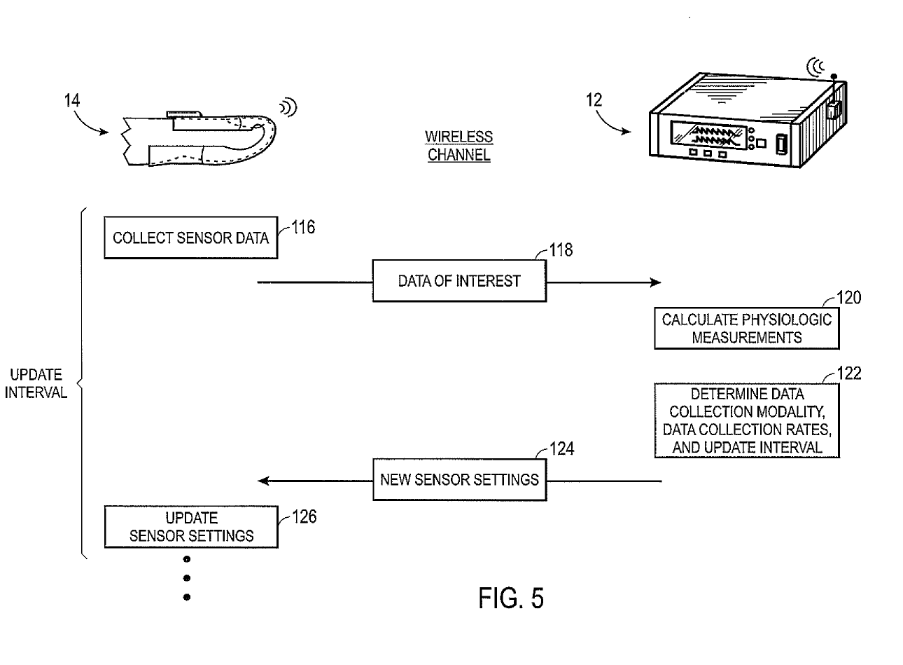

Express instructions 114 received by the wireless sensor 14 from the patient monitor 12 may constitute an eleventh example factor of the update factors 92. As described below with reference to FIG. 5, in the course of wireless communication with the sensor device 14, the patient monitor 12 may transmit updates to the wireless sensor 14 settings. These wireless sensor setting updates from the patient monitor 12 may instruct the wireless sensor 14 which subsensors in wireless sensor 14 may be used in the data collection modality (i.e., which subsensors will be actively sensing and the respective sensing functionality), the data collection rate that may be used to collect data from each subsensor, and the update interval at which the next set of subsensor settings may be derived, and others. To provide one example, a caregiver may instruct the patient monitor 12 to instruct the wireless sensor 14 to transmit a raw or processed stream of data from all of the subsensors included on the wireless sensor 14, from a specified subset of the subsensors included on the wireless sensor 14, or from a particular subsensor included on the wireless sensor 14.

With the foregoing in mind and turning to FIG. 5, a communication diagram is provided depicting communication between the wireless sensor 14 and the patient monitor 12. As mentioned above, the patient monitor 12 may be used because the monitor 12 may aid in saving the battery life of the wireless sensor 14 by taking over certain computations such as the derivation of the optimum number of subsensors to have in a data collection modality, the subsensor data collection rates, and the update interval. Additionally, the patient monitor 12 may provided greater processing power, which may allow for faster and/or more precise calculations of various medical measurements such as pulse transit times, pH, Doppler frequency calculations, SpO₂, and others. Accordingly, the wireless sensor 14 may collect (block 116) data and periodically send (block 118) data of interest for further processing by the patient monitor 12.

In one embodiment, the collected sensor data may include raw sensor data from the subsensors in the current data collection modality (i.e., the set of subsensors that are currently configured to be in an active state). The raw data may be sent to the patient monitor 12 for further processing (block 120) into discrete measurements. For example, the raw photoplethysmographic data consisting of a stream of light signal values detected by a light detector may be sent as the data of interest to the patient monitor 12 and processed by the monitor 12 to arrive at a pulse transit time. In another embodiment, only some of the collected raw data may be sent as the data of interest to the patient monitor 12. In this embodiment, the subset of the collected raw data sent to the patient monitor 12 may include only raw data that requires processing by a more powerful processor, for example, Doppler data used to create ultrasound visualizations. In yet another embodiment, already processed data (i.e., discrete patient measurements such as temperature or pulse rate values) may be sent from the wireless sensor 14 to the patient monitor 12. In this embodiment, the wireless sensor 14 may first collect the raw data, process the raw data into discrete measurements, and send the discrete measurements as the data of interest to the patient monitor 12.

In embodiments that transmit raw data, the patient monitor 12 may use the raw data sent by the wireless sensor 14 to calculate (block 120) various physiologic parameters such as pulse rate, SpO₂, microcirculation, and others. As mentioned above with respect to FIGS. 3 and 4, in certain embodiments, the monitor 12 may be used to determine the data collection modality (i.e., the subsensors and sensing functions that will be actively sensing patient conditions), the data collection rate for each subsensor in the data collection modality, and/or the next update interval. The patient monitor 12 may use the update factors 92 as discussed herein to determine (block 122) each of the subsensors that will be active in the next data collection modality, the data collection rate for each of the subsensors, and/or the next update interval for the wireless sensor 14. Once the patient monitor 12 has derived the data collection modality, the data collection rates, the next update interval, and/or other wireless sensor 14 settings, the patient monitor 12 may transmit (block 124) the new wireless sensor settings to the wireless sensor 14. The wireless sensor 14 may then update (block 126) the wireless sensor 14 settings so that the new data collection modality, data collection rates, and update intervals are used. The communication process of depicted in FIG. 5 may be repeated as needed, such as while the wireless sensor 14 is in use on a patient.

Turning to FIG. 6, a method is depicted of periodically collecting raw data from all the subsensors and all of the subsensor functionality in the wireless sensor 14 of FIG. 2. In this embodiment, raw data from all the subsensors included in the wireless sensor 14 of FIG. 2 is collected (block 128). The raw data is evaluated (block 130). As mentioned above, the evaluation of the raw data may be performed by the microprocessor 38 of the wireless sensor 14 of FIG. 2 and/or the monitor 12. Part of the evaluation process may include conversion of the raw data into discrete values such as a temperature value, a SpO₂ measurement, a pulse rate, and others. For example, the microprocessor 38 and/or the monitor 12 may process a raw photoplethysmographic data set and derive a pulse rate, respiration rate data, blood oxygen saturation data, etc. In a subsequent decision 132, the microprocessor 38 and/or the monitor 12 may evaluate whether the update factor 92 (shown in FIG. 4) circumstances have changed. By way of example, if a wireless sensor device includes a temperature subsensor and a spectrophotometric subsensor, and the temperature and spectrophotometric calculations derived from the sensors have remained stable over the recent historical period (e.g., 5 minutes) then the circumstances remain unchanged.

If circumstances remain unchanged, the process may wait (block 134) for a periodic interval. The process may then repeat after the periodic time interval has elapsed. If the circumstances have changed, the microprocessor 38 and/or the monitor 12 may determine (block 136) a new data collection modality, new data collection rates, and/or a new update interval as described with respect to FIGS. 3, 4, and 5 above. A new periodic time interval may also be determined. It is to be understood that the periodic time interval and the update interval that may be determined at block 136 need not be the same interval. By periodically querying all of the sensors in the wireless sensor device 14, the embodiment may be capable of employing the complete set of functionality present in the wireless sensor 14 device but at the same time reducing the energy usage of the device. This method allows for the use of all of the sensing functionality present in a given medical sensor in a periodic fashion.

While the disclosure may be susceptible to various modifications and alternative forms, specific embodiments have been shown by way of example in the drawings and have been described in detail herein. However, it should be understood that the embodiments provided herein are not intended to be limited to the particular forms disclosed. Indeed, the various embodiments may cover all modifications, equivalents, and alternatives falling within the spirit and scope of the disclosure as defined by the following appended claims.

What is claimed is:

1. A method for operating a wireless sensor, comprising:
operating one or more subsensors of a wireless medical sensor disposed on a patient such that at least one of the one or more subsensors do not operate continuously;
acquiring data from the one or more subsensors when operated;
determining whether to alter the operation of the one or more subsensors based on one or more factors related to at least one of the acquired data, the patient, the wireless medical sensor, or a monitor in communication with the wireless medical sensor; and
altering operation of the one or more subsensors if so determined.

2. The method of claim 1, wherein altering the operation of the at least one of the one or more subsensors comprises turning on or off one of the one or more subsensors.

3. The method of claim 1, wherein altering the operation of the at least one of the one or more subsensors comprises changing a collection rate of at least one of the one or more subsensors.

4. The method of claim 1, wherein altering the operation of the at least one of the one or more subsensors comprises changing an update interval associated with determining whether to alter the operation of the one or more subsensors.

5. The method of claim 1, wherein the one or more factors related to the acquired data comprise one or more of physiological measurements derived from the acquired data, a measure of data variability or deviation, or a trend in the acquired data or a physiological measurement derived from the acquired data.

6. The method of claim 1, wherein the one or more factors related to the patient comprise a medical history of the patient, a patient diagnosis, movement of the patient, a patient setting, or the presence or absence of a caregiver relative to the patient.

7. The method of claim 1, wherein the one or more factors related to the wireless medical sensor comprise an initialization state of the wireless medical sensor, a battery life of the wireless medical sensor, or a setting of the wireless medical sensor.

8. The method of claim 1, wherein the one or more factors related to the monitor comprise a setting of the monitor or one or more instructions input to the monitor.

9. A method comprising:
determining a data collection modality for a wireless medical sensor comprising a plurality of subsensor types, wherein the data collection modality comprises a set of the plurality of subsensor types that are selected for activation;
determining a data collection rate for each subsensor type of the data collection modality;
operating those subsensor types of the data collection modality at the data collection rate specified for each respective subsensor type; and
updating the data collection modality after an update interval.

10. The method of claim 9, wherein the plurality of subsensor types comprises at least two of a photoplethysmographic sensor, a respiration sensor, a temperature sensor, a blood pressure sensor, an electrocardiogram (EKG) sensor, a Doppler sensor, a pH sensor, a photon density sensor, a pulse transit time sensor, or any combination thereof.

11. The method of claim 9, wherein determining one or more of a data collection modality, a data collection rate, or a data collection modality and a data collection rate comprises evaluating one or more update factors.

12. The method of claim 11, wherein the one or more update factors comprise a factor related to the acquired data, the patient, the wireless medical sensor, or a monitor in communication with the wireless medical sensor.

13. The method of claim 9, comprising determining the update interval based at least on the evaluation of one or more update factors.

14. The method of claim 9, wherein determining the data collection modality, the data collection rate, or the data collection modality and the data collection rate is performed by the wireless medical sensor, by a monitor in communication with the wireless medical sensor, or by a combination of the monitor and the wireless medical sensor.

15. The method of claim 9, comprising determining a periodic interval associated with the operation of all the subsensor types.

16. The method of claim 15, wherein determining the periodic interval comprises evaluating one or more update factors.

17. A monitoring system, comprising:
a wireless medical sensor, comprising:
a battery;
a plurality of subsensors configured to draw power from the battery, wherein each subsensor is configured to acquire different types of physiological data;
one or more processors configured to execute code that operates the plurality subsensors such that the plurality of sensors is operated discontinuously;
a memory configured to store the code executed by the processor;
a wireless communication component capable of communicating the physiological data.

18. The monitoring system of claim 17, wherein the plurality of subsensors is operated discontinuously by turning off one or more of the plurality of subsensors.

19. The monitoring system of claim 17, wherein the plurality of subsensors is operated discontinuously by specifying a data collection rate for one or more of the plurality such that data is not collected continuously.

20. The monitoring system of claim 17, comprising a monitor in wireless communication with the wireless medical sensor.
